(19) United States
(12) Patent Application Publication
(10) Pub. No.: US 2009/0041715 A1
(43) Pub. Date: Feb. 12, 2009

(54) Epothilone Combinations

(75) Inventors: Richard Paul Becker, Chester, NJ (US); Anandhi Ranganathan Johri, Short Hills, NJ (US); Paul M.J. McSheehy, Loerrach-Stetten (DE); Sara Zaknoen, Hoboken, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 11/658,357

(22) PCT Filed: Jul. 25, 2005
(86) PCT No.: PCT/EP2005/008076

§ 371 Date: Aug. 22, 2008

(30) Foreign Application Priority Data
Jul. 26, 2004 (US) 60591225

(51) Int. Cl. A61K 38/20; A61K 31/427; A61K 31/282; A61K 31/505

(52) U.S. Cl. 424/85.2; 514/365; 514/492; 514/269

(57) ABSTRACT

The invention relates to combinations of (a) an epothilone with (b) two or more other antineoplastic agents for simultaneous, separate or sequential use, in particular for the delay of progression or treatment of a proliferative disease.

The invention relates to combinations of (a) an epothilone with (b) two or more other antineoplastic agents for simultaneous, separate or sequential use, in particular for the delay of progression or treatment of a proliferative disease; to a method of treating a warm-blooded animal, especially a human, having a proliferative disease comprising administering to the animal a combination which comprises (a) an epothilone and (b) two or more other antineoplastic agents; a pharmaceutical composition comprising such a combination; the use of such a combination for the preparation of a medicament for the delay of progression or treatment of a proliferative disease; and to a commercial package or product comprising such a combination.

Despite the widespread use of Taxol® and Taxotere® in the treatment of many different tumor types, the impact of taxanes on patient survival has been modest, and the overwhelming majority of metastatic tumors remain incurable. Taxane treatment is associated with a number of significant side-effects, such as peripheral neuropathy and stomatitis, and the effectiveness of taxanes can be severely limited by rapidty-developing drug resistance mechanisms, possibly involving tubulin mutations or overexpression of phosphoglycoproteins that function as drug efflux pumps. In view of these limitations as well as in view of the side-effects commonly observed with standard combination therapies, there is clearly a need for the identification of novel combinations that exhibit an improved overall profile including a broader spectrum of anti-tumor activity, efficacy against multi-drug resistant tumors and higher safety and tolerability.

In view of the relatively high toxicities associated with the treatment of proliferative diseases, by chemotherapeutics such as those mentioned above, it remains a goal to devise novel treatment schedules or novel combinations that in principle allow for treatment with lower doses of the individual compounds, thus making it possible to allow for diminuation of the toxicities individually associated with highly toxic compounds. In addition, novel treatment regimens and combinations allowing for improved efficiency in the treatment of proliferative diseases remain an ever existing need. Furthermore, specific proliferative diseases and/or specific patient groups (e.g. related to sex or especially age, such as in case of pediatric or geriatric use, or patients where the proliferating cells became refractory to treatment with known chemotherapeutics or combinations thereof) may require more specific, even individual therapeutic regimens.

The microtubule-stabilizing effect of the epothilones was first described by Bollag et al., Cancer Research 55, 1995, 2325-33. A suitable treatment schedule of different types of tumors, especially tumors which are refractory to the treatment by other chemotherapeutics, in particular TAXOL™, is described in WO 99143320. It has now been found the epothilones, especially Epothilone A or B, in combination with two or more antineoplastic agents is effective in the treatment of proliferative diseases.

### GENERAL DESCRIPTION OF THE INVENTION

Surprisingly, it has now been found that the combination of (a) an epothilone with (b) two or more other antineoplastic agents for simultaneous, separate or sequential use, in particular for the delay of progression or treatment of a proliferative disease, shows many of the advantages mentioned above as desirable.

Unexpectedly, it has been found that the antineoplastic effect, i.e. especially the delay of progression or treatment of a proliferative disease, in particular the treatment of a tumor or more particularly of a solid tumor, by combination as defined herein is greater than the effects that can be achieved with either type of combination partner alone, i.e. greater than the effects of a therapy using only component (a) or the two or more combination partners of component (b) as defined herein. A further benefit is that lower doses of the active ingredients can be used, for example, that the dosages need not only often be smaller but are also applied less frequently, or can be used in order to diminish the incidence of side-effects, thus allowing an improved quality of life, a decreased mortality and/or a decreased morbidity. This is in accordance with the desires and requirements of the patients to be treated.

In particular, combinations of the present invention can be shown to be synergistic, thus allowing improved therapeutic efficiency and/or lower dosing of the individual components.

### DETAILED DESCRIPTION OF THE INVENTION

In one preferred embodiment, the invention relates to a combination of (a) an epothilone and (b) two or more other antineoplastic agents for simultaneous, separate or sequential use, in particular for use in the delay of progression or the treatment of a proliferative disease in a warm-blooded animal, especially a human.

In another preferred embodiment, the invention relates to a method of treating a warm-blooded animal, especially a human, suffering from a proliferative disease, comprising administering to said animal a combination which comprises (a) an epothilone and (b) two or more other antineoplastic agents, preferably in such a way that the components (a) and (b) are jointly therapeutically active in the treatment of said disease; in particular in a dose that is pharmaceutically effective in the treatment of said disease.

A further embodiment of the invention relates to a pharmaceutical composition comprising a combination of (a) an epothilone with (b) two or more other antineoplastic agents and optionally at least one pharmaceutically acceptable carrier, preferably for simultaneous, separate or sequential use, especially in the delay of progression or treatment of a proliferative disease in a warm-blooded animal, especially a human, requiring such treatment.

Still a further embodiment of the invention relates to the use of a combination of (a) an epothilone with (b) two or more other antineoplastic agents for simultaneous, sequential or separate use, for the delay of progression or the treatment of a proliferative disease; and/or for the manufacture of a pharmaceutical preparation for the delay of progression or treatment of said disease.

Yet another embodiment of the invention relates to a commercial package or product comprising (a) an epothilone and (b) two or more other antineoplastic agents for simultaneous, chronically staggered or (less preferably) separate use, especially for the delay of progression or the treatment of a proliferative disease.

The general terms used hereinbefore and hereinafter preferably have within the context of this disclosure the following meanings, unless otherwise indicated:

As components (a) and (b), the following are preferred:

Component (a) is preferably an epothilone derivative of formula I

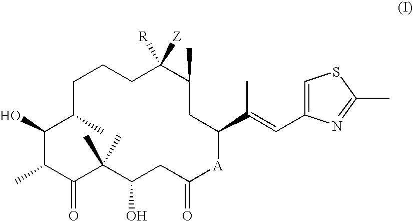

wherein A represents O or NRN, wherein RN is hydrogen or lower alkyl, R is hydrogen or lower alkyl, and Z is O or a bond.

A compound of formula I wherein A represents O, R is hydrogen and Z is O is known as epothilone A; a compound of formula I wherein A represents O, R is methyl and Z is O is known as epothilone B; a compound of formula I wherein A represents O, R is hydrogen and Z is a bond is known as epothilone C; a compound of formula I wherein A represents O, R is methyl and Z is a bond is known as epothilone D.

Epothilone derivatives of formula I wherein A represents O or NRN, wherein RN is hydrogen or lower alkyl, R is hydrogen or lower alkyl and Z is O or a bond, and methods for the preparation of such epothilone derivatives are in particular generically and specifically disclosed in the patents and patent applications WO 93/10121, U.S. Pat. No. 6,194,181, WO 98/25929, WO 98/08849, WO 99/43653, WO 98/22461 and WO 00/31247 in each case in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding crystal modifications, e.g. solvates and polymorphs, which are disclosed therein. Epothilone derivatives of formula I, especially epothilone B, can be administered as part of pharmaceutical compositions which are disclosed in WO 99/39694.

The transformation of epothilone B to the corresponding lactam is disclosed in Scheme 21 (page 31, 32) and Example 3 of WO 99/02514 (pages 48-50). The transformation of a compound of formula I which is different from epothilone B into the corresponding lactam can be accomplished analogously. Corresponding epothilone derivatives of formula I wherein RN is lower alkyl can be prepared by methods known in the art such as a reductive alkylation reaction starting from the epothilone derivative wherein RN is hydrogen.

Especially preferred are Epothilone A and/or B with Epothilone B most preferred as component (a).

Component (b) preferably comprises two or more, more preferably two or three, most preferably two antineoplastic agents other than component (a), in the latter case leading to a triple drug combination.

The term “antineoplastic agents” as used herein includes, but is not limited to topoisomerase I inhibitors, topoisomerase II inhibitors, other microtubule active agents, alkylating agents, antineoplastic antimetabolites, platin compounds, angiostatic steroids, biological response modifiers, monoclonal antibodies, proteasome inhibitors, EGFR inhibitors, leucovorin, interleukin, temozolomide and hexylmethyl-melamine, all independently in free form or as pharmaceutically acceptable salts.

The term “topoisomerase I inhibitor”, as used herein, includes, but is not limited to, topotecan, irinotecan, camptothecian and its analogues, 9-nitrocamptothecin and the macromolecular camptothecin conjugate PNU-166148 (compound Al in WO 99117804). Irinotecan can be administered in the form as it is marketed, e.g., under the trademark CAMPTOSAR. Topotecan can be administered in the form as it is marketed, e.g., under the trademark HYCAMTIN.

The term “topoisomerase II inhibitors” as used herein includes, but is not limited to the antracyclines such as doxorubicin, epirubicin, idarubicin and nemorubicin, the anthraquinones mitoxantrone and losoxantrone, and the podophyllotoxines etoposide and teniposide. Etoposide can be administered in the form as it is marketed, e.g. under the trademark ETOPOPHOS™. Teniposide can be administered in the form as it is marketed, e.g. under the trademark VM 26-BRISTOL™. Doxorubicin can be administered, e.g., in the form as it is marketed, e.g. under the trademark ADRIBLASTIN™ or the liposomal formulation CAELYX™ or DOXIL. Epirubicin can be administered in the form as it is marketed, e.g. under the trademark FARMORUBICIN™. Idarubicin can be administered in the form as it is marketed, e.g. under the trademark ZAVEDOS™. Mitoxantrone can be administered in the form as it is marketed, e.g. under the trademark NOVANTRON™, Doxorubican and mitoxantrone are preferred.

The term “other microtubule active agent” refers to microtubule active agents other than epothilones especially microtubule stabilizing, microtubule destabilizing agents and microtublin polymerization inhibitors including, but not limited to, taxanes, e.g., paclitaxel and docetaxel; vinca alkaloids, e.g., vinblastine, especially vinblastine sulfate; vincristine, especially vincristine sulfate and vinorelbine; discodermolides; and cochicine. Paclitaxel may be administered, e.g., TAXOL; docetaxel as TAXOTERE; vinblastine sulfate as VINBLASTIN R.P; and vincristine sulfate as FARMISTIN. Discodermolide can be obtained, e.g., as disclosed in U.S. Pat. No. 5,010,099. Included herewith are also oral taxane formulations.

The term “alkylating agent”, as used herein, includes, but is not limited to, cyclophosphamide, ifosfamide, melphalan, Dacarbazine (or DTCI), or nitrosourea (BCNU or Gliadel). Cyclophosphamide can be administered in the form as it is marketed, e.g., under the trademark CYCLOSTIN; and ifosfamide as HOLOXAN. Cyclophosphamide is preferred.

The term “anti-neoplastic anti-metabolite” includes, but is not limited to, 5-fluorouracil (5-FU); capecitabine; gemcitabine; DNA de-methylating agents, such as 5-azacytidine and decitabine; methotrexate; and edatrexate. Capecitabine can be administered in the form as it is marketed, e.g., under the trademark XELODA; and gemcitabine as GEMZAR. Also included is the monoclonal antibody trastuzumab which can be administered in the form as it is marketed, e.g., HERCEPTIN.

The term “folinic acid” relates to “N-[4-[[(2-amino-5-formyl-1,4,5,6,7,8-hexahydro-4-oxo-⁶-pteridinyl)methyl]amino]benzoy]-L-glutamic acid, which is marketed, e.g., under the trademark LEUCOVORINT™.

The term “platin compound”, as used herein, includes, but is not limited to, carboplatin, cis-platin, cisplatinum and oxaliplatin. Carboplatin can be administered in the form as it is marketed, e.g., CARBOPLAT; and oxaliplatin as ELOXATIN.

“Angiostatic steroids”, as used herein, refers to agents which block or inhibit angiogenesis, such as, anecortave, triamcinolone, hydrocortisone, 11-α-epihydrocotisol, cortexolone, 17α-hydroxyprogesterone, corticosterone, desoxycorticosterone, testosterone, estrone and dexamethasone.

The term “proteasome inhibitor”, as used herein, includes, but is not limited to, bortezomib or Velcade.

The term “biological response modifier”, as used herein, refers to a lymphokine or interferons, e.g., interferon γ.

The term “monoclonal antibody”, as used herein, includes, but is not limited to, bevacizumab and cetuximab. Bevacizumab can be administered in the form as it is marketed, e.g., AVASTIN; and cetuximab as ERBITUX.

The term “EGFR inhibitor”, as used herein, includes, but is not limited to, erlotinib and gefitinib. Erlotinib can be administered in the form as it is marketed, e.g., TARCEVA; and Gefitinib as IRESSA.

Additional neoplastic agents for use in combinations of the present invention include temozolomide which can be administered in the form as it is marketed, e.g., TEMODAR and hexylmethyl-melamine can be administered in the form as it is marketed, e.g. ALTRETAMINE.

In each case where citations of patent applications or scientific publications are given, in particular with regard to the respective compound claims and the final products of the working examples therein, the subject-matter of the final products, the pharmaceutical preparations and the claims is hereby incorporated into the present application by reference to these publications. Comprised are likewise the corresponding stereoisomers as well as the corresponding crystal modifications, e.g. solvates and polymorphs, which are disclosed therein. The compounds used as active ingredients in the combinations disclosed herein can be prepared and administered as described in the cited documents, respectively.

The structure of the active agents identified by code numbers, generic or trade names may be taken from the actual edition of the standard compendium “The Merck Index” or from databases, e.g. Patents International (e.g. IMS World Publications), or the publications mentioned above and below. The corresponding content thereof is hereby incorporated by reference.

It will be understood that references to the components (a) and (b) are meant to also include the pharmaceutically acceptable salts of any of the active substances (microtubule active agent or antineoplastic agent) comprised. If active substances comprised by components (a) and/or (b) have, for example, at least one basic center, they can form acid addition salts. Corresponding acid addition salts can also be formed having, if desired, an additionally present basic center. Active substances having an acid group (for example COOH) can form salts with bases. The active substances comprised in components (a) and/or (b) or a pharmaceutically acceptable salts thereof may also be used in form of a hydrate or include other solvents used for crystallization. Epothilone A or B, i.e. the most preferred combination partner (a), is preferably Epothilone B.

A proliferative disease is mainly a tumor disease (or cancer) (and/or any metastases), wherever the tumor or the metastasis are located), more especially a tumor selected from the group comprising breast cancer, genitourinary cancer, lung cancer, gastrointestinal cancer, epidermoid cancer, melanoma, ovarian cancer, pancreas cancer, neuroblastoma, head and neck cancer (this term, wherever it is used, meaning a head and/or neck cancer, meaning that not only a cancer of head and neck, but also of head or neck is envisaged) or bladder cancer, or in a broader sense renal, brain or gastric cancer; more preferably (i) a tumor selected from a breast tumor; an epidermoid tumor, especially and epidermoid head and neck, preferably mouth, tumor; and a lung tumor, especially a non-small cell lung tumor; or from a gastrointestinal tumor, especially a colorectal tumor; and a genitourinary tumor, especially a prostate tumor (especially a hormone-refractory prostate tumor); or (ii) (more preferably) a proliferative disease that is refractory to the treatment with other chemotherapeutics, especially a corresponding tumor (and/or any metastasis), more especially a tumor selected from the group comprising tumors that are refractory to a standard treatment with (an)other chemotherapeutic(s), especially with 5-fluorouracil and/or (preferably) a microtubule stabilizing agent of the taxane class, most especially TAXOL®, still more preferably a tumor selected from gastrointestinal, e.g. colorectal (especially refractory to standard, e.g. 5-fluorouracil, and/or TAXOL treatment); and genitourinary, e.g. prostatic tumors (and/or a metastasis thereof, especially a metastasis thereof); most preferably a gastrointestinal tumor, especially a colorectal cancer; or (iii) a tumor that is refractory to treatment with other chemotherapeutics due to multidrug resistance, especially refractory to a member of the taxane class of microtubule stabilizing agents, preferably TAXOL®, most especially a multidrug, especially TAXOL®, resistant lung tumor (especially a non-small cell lung tumor), a multidrug resistant breast tumor, or a multidrug resistant epidermoid, preferably epidermoid head and neck, most preferably mouth, tumor.

Combinations of the present invention can be used in first, second and subsequent lines of therapy; to allow use of the combination preceding and/or following any chemotherapy, any radiotherapy or any surgery, for primary and metastatic disease.

In a broader sense of the invention, a proliferative disease may furthermore be selected from hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Where hereinbefore and subsequently a tumor, a tumor disease, a carcinoma or a cancer are mentioned, also metastasis in the original organ or tissue and/or in any other location are implied alternatively or in addition, whatever the location of the tumor and/or metastasis is.

The word “refractory” means that the respective proliferative disease (especially a tumor and/or any metastasis thereof), upon treatment with a (meaning at least one) chemotherapeutic other than an epothilone, shows no or only weak antiproliferative response (no or only weak inhibition of tumor growth) after the treatment with such an agent, that is, a tumor that cannot be treated at all or only with unsatisfying results with other (preferably standard) chemotherapeutics (preferably as defined above, especially 5-fluorouracil (especially in the case of colorectal, like colon, cancer), antiandrogens or preferably mitoxantrone and Taxotere (especially in the case of prostate cancer), or antiestrogens, like letrozole (especially in the case of breast cancer); or especially a member of the taxane class of chemotherapeutics, e.g. TAXOTERE® or TAXOL®, in a warm-blooded animal, especially a human; for example the tumor growth is not stopped, only retarded slightly or no regression is found. The present invention, where treatment of refractory tumors and the like is mentioned, is to be understood to encompass not only (a) tumor(s) where one or more chemotherapeutics have already failed during treatment of a patient, but also (a) tumor(s) that can be shown to be refractory by other means, e.g. biopsy and culture in the presence of chemotherapeutics. Where a term like “refractory against TAXOL®” is used hereinbefore and hereinafter, this term, in addition to the finished product, is also intended to mean paclitaxel, the active substance of TAXOL®.“Refractory to hormone treatment” or “hormone refractory”, in the case of a tumor of the genitourinary tract, especially a prostate tumor, means refractory to treatment with an antiandrogen.

TAXOL® preferably stands for the finished product that comprises paclitaxel, but, in a broader sense, is also meant to encompass paclitaxel itself of any other paclitaxel formulation with one or more carrier material(s).

Preferably, the term refractory means that with standard dose a reduction of tumor growth by less than 50% (that is a T/C% value of equal to or more than 50%) is obtained when compared with a control without chemotherapeutic, e.g. by in vivo or in vitro measurements.

Multidrug resistant tumor disease is one where resistance to one or more chemotherapeutics, including those of the taxane class, especially TAXOL®, or the anthracycline class, especially ADRIAMYCIN®, is found. The basis for this resistance is the export via an energy (especially ATP)-dependent pump located on the surface of cells of the respective tumor, especially of the P-glycoprotein family, especially P-glycoprotein (P-gp) itself. In the present invention, alternatively or in addition other mechanisms may cause a tumor to be refractory to treatment with chemotherapeutics other than an epothilone. For example, alterations in the drug target (especially microtubules in the present case), changes in the intracellular metabolism that may inactivate the compound, or changes in the physiology of the cell that would facilitate by-passing or overriding of the mechanism of drug action may lead to such resistance.

Simultaneous administration may, for example, take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points, preferably meaning that the components (a) and (b) are administered such that no overlap of measurable blood levels of both compounds are present in an overlapping manner (at the same time).

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

Accordingly, the preparations according to the inventions may be fixed combinations of (a) an epothilone and (b) two or more other antineoplastic agents, or combinations of more than one separate pharmaceutical preparations each comprising one (or more than one) of these active ingredients in separate form (e.g. in the sense of a kit of parts).

In a yet further aspect, the present invention provides a pharmaceutical preparation comprising (a) an epothilone and (b) two or more other antineoplastic agents, together with a pharmaceutically acceptable carrier.

The term “delay of progression” as used herein means administration of the combination to patients being in a pre-stage or in an early phase, of the first manifestation or a relapse of the disease to be treated, in which patients for example a pre-form of the corresponding disease is diagnosed or which patients are in a condition, e.g. during a medical treatment or a condition resulting from an accident, under which it is likely that a corresponding disease will develop.

“Jointly therapeutically active” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect). Whether this is the case, can inter alia be determined by following the blood levels, showing that both compounds are present in the blood of the human to be treated at least during certain time intervals.

“Pharmaceutically effective” preferably relates to an amount that is therapeutically or in a broader sense also prophylacticly effective against the progression of a proliferative disease, especially a solid tumor, preferably one as defined above.

The term “a commercial package” or “a product”, as used herein defines especially a “kit of parts” in the sense that the components (a) and (b) as defined above can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods, e.g. the determination of Combination Index or the use of isobolograms as described in the examples). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g. less side effects or a combined therapeutical effect in a non-effective dosage of one or both of the combination partners (components) (a) and (b), and very preferably a strong synergism (Combination Index above 4) of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically (e.g. more than 3 to 4 weeks of daily dosing) at a later time point and subsequently the other component or the combinaton of both components at a still later time point (in subsequent drug combination treatment courses for an optimal antitumor effect) or the like.

Any of the combination of components (a) and (b), the method of treating a warm-blooded animal comprising administering these components, a pharmaceutical composition comprising these two components for simultaneous, separate or sequential use, the use of the combination for the delay of progression or the treatment of a proliferative disease or for the manufacture of a pharmaceutical preparation for these purposes or a commercial product comprising such a combination of components (a) and (b), all as mentioned or defined above, will be referred to subsequently also as COMBINATION OF THE INVENTION (so that this term refers to each of these embodiments which thus can replace this term where appropriate).

It can be shown by established test models and in particular those test models described herein, e.g. in the Examples, that a COMBINATION OF THE INVENTION results in a more effective delay of progression or treatment of a proliferative disease compared to the effects observed with the single combination partners or combination according to component (b) only (two or more antineoplastic agents other than an epothilone). The person skilled in the pertinent art is fully enabled to select a relevant test model to prove the therapeutic indications and beneficial effects hereinbefore and hereinafter mentioned. The pharmacological activity of a COMBINATION OF THE INVENTION may, for example, be demonstrated in a clinical study or in a test procedure as essentially described hereinafter.

Suitable clinical studies are, for example, open label non-randomized, dose escalation studies (Phase I) in patients with advanced solid tumors. Such studies prove (A) safety and (B) the synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy or a therapy using only two or more antineoplastic agents other than an epothilone (component (b)) versus a COMBINATION OF THE INVENTION. Preferably, the combination partner (a) is administered with a fixed dose and the dose of the combination partner (b) is escalated until the Maximum Tolerated Dosage of the combination regimen is reached, or vice versa. In a preferred embodiment of the study, each patient receives daily doses of the combination partner (a). Alternatively, a placebo-controlled, double blind study can be used in order to prove the benefits of the COMBINATION OF THE INVENTION mentioned herein, once the safety of the treatment regimen(s) has been established.

The COMBINATION OF THE INVENTION can also be applied in combination with other treatments, e.g. surgical intervention, hyperthermia and/or irradiation therapy.

### PREFERRED EMBODIMENTS OF THE INVENTION

In the following preferred embodiments of the invention, more general terms can be replaced independently or totally by the more specific definitions given above, thus leading to still more preferred embodiments of the invention.

A COMBINATION OF THE INVENTION which comprises (a) Epothilone A or B or a pharmaceutically acceptable salt thereof, and (b) at least two further antineoplastic agents, independently in free form or as pharmaceutically acceptable salts, preferably as defined above, is preferred.

More preferred is a COMBINATION OF THE INVENTION comprising (a) Epothilone A or B, or a pharmaceutically acceptable salt thereof, and (b) two or three, preferably two, further antineoplastic agents selected from topoisomerase I inhibitors, topoisomerase II inhibitors, other microtubule active agents, alkylating agents, antineoplastic antimetabolites, platin compounds, angiostatic steroids, biological response modifiers, monoclonal antibodies, proteasome inhibitors, EGFR inhibitors, leucovorin, interleukin, temozolomide and hexylmethyl-melamine, which are independently of each other in free form or as pharmaceutically acceptable salts.

Still more preferred is a COMBINATION OF THE INVENTION comprising (a) Epothilone B or a pharmaceutically acceptable salt thereof, and (b) two further antineoplastic agents selected from topoisomerase I inhibitors, topoisomerase II inhibitors, other microtubule active agents, alkylating agents, antineoplastic antimetabolites, platin compounds, angiostatic steroids, biological response modifiers, monoclonal antibodies, proteasome inhibitors, EGFR inhibitors, leucovorin, interleukin, temozolomide and hexylmethyl-melamine, which are independently of each other in free form or as pharmaceutically acceptable salts.

Most preferably the invention relates to a COMBINATION OF THE INVENTION comprising (a) Epothilone B, or a pharmaceutically acceptable salt thereof, and (b) one two further antineoplastic agents, especially selected from Vincristine, Vinblastine, Vinorelbine, Topotecan, Irinotecan, capecitabine, Etoposide, Doxil, doxirubican, Mitoxantrone, Cyclophosphamide, Cisplatinum, Carboplatin, Oxaliplatin, Herceptin, Leucovorin, Radiation, Prednisone, Interluekin, Interferon, Estramustine, 5FU/LV, Dacarbazine, bortezormib, bevacizumab, cetuximab, erlotinib, gefitinib, temozolomide and hexylmethyl-melamine, Gemcitabine, or Procarbazine, which are independently of each other in free form or as pharmaceutically acceptable salts.

In a further embodiment of the present invention the combination is such that administration of component (a) is started before administration of component (b), especially 2 to 48 hours before.

Another preferred embodiment comprises Epothione A or B; a second microtubule agent e.g. vincristine or vinorelbine; and a third agent selected from antineoplastic antimetabolites, alkylating agents, monoclonal antibodies or a platinum compound. In this embodiment the third agent can be procarbazine, herceptin, estramustine, cisplatinum or gemcitabine.

Another embodiment comprises Epothione A or B; a platinum compound; and a third agent selected from antineoplastic antimetabolites, topoisomerase II inhibitors or radiation. In this embodiment the platinum compound can be cisplatinum, oxaliplatin or carboplatinum. In this embodiment the third agent can be gemcitabine, capecitabine, 5FU/LV, doxirubican or etoposide.

Another embodiment comprises Epothione A or B; a topoisomerase II inhibitor; and a third agent selected from antineoplastic antimetabolites, alkylating agents or an angiostatic steroid. In this embodiment the topoisomerase II inhibitor can be doxorubicin or mitoxantrone, and the third agent can be cyclophosphamide, herceptin, estramustine or prednisone.

In any of the preceding paragraphs describing preferred embodiments of the invention, those COMBINATIONS OF THE INVENTION are most preferred where the active compounds used in component (a) and component (b) are formulated independently or in the form of a kit of parts, in both cases based on pharmaceutical preparations that are already (e.g. commercially) available.

### Pharmaceutical Preparations and Methods

The pharmaceutical preparations comprising component (a) and/or component (b), in the case of component (b) a fixed combination of the antineoplastic agents comprised therein or independent formulations for one or more of these antineoplastic agents for combined use, can be standard preparations of these components as already known in the art.

The pharmaceutical compositions comprise from about 0.00002 to about 95%, especially (e.g. in the case of infusion dilutions that are ready for use) of 0.0001 to 0.02%, or (for example in case of injection or infusion concentrates or especially parenteral formulations) from about 0.1% to about 95%, preferably from about 1% to about 90%, active ingredient (weight by weight, in each case). Pharmaceutical compositions according to the invention may be, for example, in unit dose form, such as in the form of ampoules, vials, dragees, tablets, infusion bags or capsules.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The microtubule active agent and further (other) antineoplastic agents forming part of components (a) and (b) are named “active ingredients” in the following definition of pharmaceutical preparations/compositions:

The pharmaceutical compositions of the present invention are prepared in a manner known per se, for example by means of conventional dissolving, lyophilizing, mixing, granulating or confectioning processes and combination with appropriate carrier materials.

Solutions of the active ingredient, and also suspensions, and especially isotonic aqueous solutions or suspensions, are useful for parenteral administration of the active ingredient, it being possible, for example in the case of lyophilized compositions that comprise the active ingredient alone or together with a pharmaceutically acceptable carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes.

The injection or infusion compositions are prepared in customary manner under sterile conditions; the same applies also to introducing the compositions into ampoules or vials and sealing the containers.

An infusion solution preferably must have the same or essentially the same osmotic pressure as body fluid. Accordingly, the aqueous medium preferably contains an isotonic agent which has the effect of rendering the osmotic pressure of the infusion solution the same or essentially the same as body fluid.

The isotonic agent may be selected from any of those known in the art, e.g. mannitol, dextrose, glucose and sodium chloride. The infusion formulation may be diluted with the aqueous medium. The amount of aqueous medium employed as a diluent is chosen according to the desired concentration of active ingredient in the infusion solution.

Infusion solutions may contain other excipients commonly employed in formulations to be administered intravenously. Excipients include antioxidants. Infusion solutions may be prepared by mixing an ampoule or vial of the formulation with the aqueous medium, e.g. a 5% w/v glucose solution in WFI or especially 0.9% sodium chloride solution in a suitable container, e.g. an infusion bag or bottle. The infusion solution, once formed, is preferably used immediately or within a short time of being formed, e.g. within 6 hours. Containers for holding the infusion solutions may be chosen from any conventional container which is nonreactive with the infusion solution. Glass containers made from those glass types aforementioned are suitable although it may be preferred to use plastics containers, e.g. plastics infusion bags.

Pharmaceutical compositions for parenteral, e.g. oral administration, can be obtained by combining the active ingredient with solid carriers, if desired granulating a resulting mixture, and processing the mixture, if desired or necessary, after the addition of appropriate excipients, into tablets, dragee cores or capsules, or be filled into a powder inhalator for administration by inhalation. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or tablet coatings, for example for identification purposes or to indicate different doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

In the case of combinations with one or more other active ingredients, a fixed combination of two or more components or two or more independent formulations (e.g. in a kit of part) are prepared as described above, or the other active ingredients are used in standard formulations that are marketed and known to the person of skill in the art, and the compound of the present invention and any other chemotherapeutic are administered at an interval that allows for a joint, especially a parallel, additional or preferably synergistic effect in the treatment of a proliferative disease, especially a solid tumor (especially as defined above).

The doses of chemotherapeutics to be combined with a long-acting beta-2 adrenoreceptor agonist are, for example, those used in standard treatment known in the art, e.g. as described in R. T. Skeel, Handbook of Cancer Chemotherapy, Fifth Edition, Lippincott Williams & Wilkins, Philadelphia et al., 1999, or, In view of the synergism, somewhat lower doses, e.g. between 5 and 60% of the dose without combination, respectively; the dose in each case depending on the status, age, sex, weight and other relevant properties of the patient. They can be formulated separately, especially being used in known pharmaceutical compositions, preferably combined as a kit comprising pharmaceutical preparations of each active compound (kit of parts), or in fixed combination.

Some examples for preferred dosages are represented in the following:

If the warm-blooded animal is a human, the dosage of a compound of formula I is preferably in the range of about 0.25 to 75, preferably 0.5 to 50, e.g. 2.5, mg/m2 once weekly for two to four, e.g. three, weeks, followed by 6 to 8 days off in the case of an adult patient.

Epothilone B is preferably administered in a dose which is calculated according to the formula (III)

single dose (mg/m2)=(0.1 to y)×N (III)

wherein N is the number of weeks between treatments and y is 6, wherein epothilone B is administered in more than one treatment cycle after an interval of one week to six weeks after the preceding treatment

In one preferred embodiment of the invention, epothilone B is administered weekly in a dose that is between about 0.1 to 6 mg/m2, preferably between 0.1 and 3 mg/m2, e.g. 2.5 or 3.0 mg/m2, for three weeks after an interval of one to six weeks, especially an interval of one week, after the preceding treatment. In another embodiment of the invention said epothilone B is preferably administered to a human every 18 to 24 days in a dose that is between about 0.3 and 12 mg/m2.

Among the topoisomerase I inhibitors, topotecan may be administered to a human in a dosage range varying from about 1 to 5 mg/m²day. Irinotecan may be administered to a human in a dosage range varying from about 50 to 350 mg/m²day.

Among the topoisomerase II inhibitors, doxorubicin may be administered to a human in a dosage range varying from about 10 to 100 mg/m2day, e.g. 25 or 75 mg/m2day, e.g. as single dose; epirubicin may be administered to a human in a dosage range varying from about 10 to 200 mg/m²day; idarubicin may be administered to a human in a dosage range varying from about 0.5 to 50 mg/m²day, e.g. 8 mg/m²day during three days; and mitoxantrone may be administered to a human in a dosage range varying from about 2.5 to 25 mg/m²day, e.g. 10-14 mg/m²day during 5 to 8 days .

Vinblastine may be administered to a human in a dosage range varying from about 1.5 to 10 mg/m²day. Vincristine sulfate may be administered parenterally to a human in a dosage range varying from about 0.025 to 0.05 mg/kg body weight·week. Vinorelbine may be administered to a human in a dosage range varying from about 10 to 50 mg/m²day. Tamoxifen citrate may be administered to a human in a dosage range varying from about 10 to 40 mg/day. Paclitaxel may be administered to a human in a dosage range varying from about 50 to 300 mg/m²day. Etoposide phosphate may be administered to a human in a dosage range varying from about 25 to 115 mg/m²day, e.g. 56.8 or 113.6 mg/m²day. Docetaxel may be administered to a human in a dosage range varying from about 25 to 100 mg/m²day. Cyclophosphamide may be administered to a human in a dosage range varying from about 50 to 1500 mg/m²day. 5-Fluorouracil may be administered to a human in a dosage range varying from about 50 to 1000 mg/m²day, e.g. 500 mg/m²day. Capecitabine may be administered to a human in a dosage range varying from about 10 to 1000 mg/m²day. Gemcitabine hydrochloride (not highly recommended as secondary malignancies may occur) may be administered to a human in a dosage range varying from about 1000 mg/week. Carboplatin may be administered to a human in a dosage range varying from about 200 to 400 mg/M² about every four weeks. Cisplatin may be administered to a human in a dosage range varying from about 25 to 75 mg/m² about every three weeks. Oxaliplatin may be administered to a human in a dosage range varying from about 50 to 85 mg/m² every two weeks. Trastuzumab may be administered to a human in a dosage range varying from about 1 to 4 mg/m²week. Leucovorin may be administered to a human in a dosage range varying from about 10 to 50 mg/M² given orally or intravenously, daily or intermittently each week. Prednisone may be administered to a human in a dosage range varying from about 5 to 100 mg given orally per day. Interleukin may be administered to a human in a dosage range varying from about 0.5 to 10 million Units given subcutaneously, intravenously (bolus and continuous over some days).lnterferon may be administered to a human in a dosage range varying from about 0.5 to 6 million Units/m² given several times a week, given subcutaneously. Estramustine may be administered to a human in a dosage range varying from about 100 to 300 mg/m² given several time a week, given orally. Procarbazine may be administered to a human in a dosage range varying from about 50 to 300 mg per day given orally. Velcade may be administered to a human in a dosage range varying from about 0.5 to 2.5 mg/M² per dose, given intravenously twice a week.

Components (a) and (b) can be prepared according to methods that are known in the art, e.g. as described in any of the references quoted herein, and/or they are commercially available. The most preferred combination partner (a), Epothilone A or B, can be prepared and administered as described in WO 99/43320.

### EXAMPLES

The following Examples serve to illustrate the invention without limiting the scope thereof:

### Example 1

Outlining of Clinical Trials with EPO906/Oxiplatin/Caoecitabine Combinations

Clinical trials are conducted with patients having advanced colon or colorectal cancer. The required permissions are obtained.

Day of Treatment Cycle

| Day 1 | Day 8 | Day 15 |
|---|---|---|
| Oxaliplatin | — | Rest |
| EPO906 | EPO906 | Rest |

| | |
|---|---|
| ←Capecitabine BID→ | Rest |
| Days 1 through 14 | |

The following dose escalation scheme will be employed:

| Dose | Oxaliplatin | EPO906 | Capecitabine |
|---|---|---|---|
| Level | mg/m2 | mg/m2 | mg/m2 BID |
| 1B | 85 | 2.0 | 500 (equivalent daily dose: 1000) |
| 2B | 104 | 2.0 | 750 (equivalent daily dose: 1500) |
| 3B | 104 | 2.5 | 1000 (equivalent daily dose: 1500) |

### Preparation of EPO906 for Administration

EPO906 is supplied in dosage strength of 5 mg/2 mL concentrate for solution for injection either in individual 10 mL glass vials or in individual 6 mL glass vials requiring two dilution steps prior to administration. The drug is formulated in polyethylene glycol 300 (PEG 300) and must be pre-diluted in 0.9% NaCl solution to obtain a concentrate of 1 mg/ml.

### Preparation of the Initial Diluted Solution

EPO906 5 mg/2 mL: Dilute the vial contents with 3.4 mL of physiological saline resulting in 5 mL (usable volume) of a 1.0 mg/mL solution of EPO906. Shake the vial gently and allow it to stand for at least 5 min to allow air bubbles formed during the dilution to separate. The pre-diluted product must be Inspected visually for particulate matter and discoloration, if either is present, a new vial must be used.

EPO906 10 mg/4 mL: Dilute the vial contents with 6.5 mL of physiological saline resulting in 10 mL (usable volume) of a 1.0 mg/mL solution of EPO906. Shake the vial gently and allow it to stand for at least 5 min to allow air bubbles formed during the dilution to separate. The pre-diluted product must be inspected visually for particulate matter and discoloration, if either is present, a new vial must be used.

### Preparation of the Final Dilution for Injection

Aseptically withdraw the required volume of the initial diluted EPO906 solution from the vial using a graduated disposable syringe. Transfer under aseptic conditions the required content from the syringe into infusion bottles/infusion bags typically containing 50 ml physiological saline (0.9% NaCl) to produce a final concentration of 0.05 mg/ml to 0.2 mg/ml EP0906. Thoroughly mix the infusion by manual rotation.

### Administration of EPO906

EPO906 should be administered intravenously over a period of 5 to 10 minutes using the best available in-dwelling line. The line should be thoroughly flushed with 4-5 ml saline immediately following the administration of EPO906. If an in-dwelling line is not available, a butterfly infusion line should be inserted and utilized, and flushed, as previously described, immediately following completion of the administration of EPO906.

### Preparation of Oxaliplatin For Bolus Administration

Oxaliplatin vials are stored at room temperature between 20-25° C. protected from light. Reconstituted solution in sterile water of 5% dextrose may be stored for 24 to 48 hours at 2 to 8° C. After further dilution in 5% dextrose, the solution is stable for 24 hours at room temperature. The freeze-dried powder is reconstituted by adding 10 ml (for the 50 mg vials) or 20 mL (for the 100 mg vials of Water for Injection or Dextrose 5% in Water) to yield a 5 mg/mL solution. The reconstituted solution must be further diluted in an infusion solution of 250 mL Dextrose 5% in Water. The reconstitution or final dilution must never be performed with a sodium chloride solution.

### Administration of Oxaliplatin

The diluted solution of oxaliplatin in 250-500 ml 5% dextrose is administered by an infusion pump over 2 hours.

### Capecitabine Administration

Capecitabine will be prescribed at the assigned treatment dose, according to schedules approved by the FDA on Apr. 30, 1998. A prescription will be given at the appropriate dose, to be taken for 14 days followed by a 1-week rest period, given as 3 week cycles. Capecitabine tables should be swallowed with water within 30 minutes after a meal.

During the first treatment cycle, blood samples will be collected in EDTA tubes for the pharmacokinetic sampling of EPO906. Blood will be drawn at the following time points:

Pharmacokinetic sampling for EPO906:

| Cycle 1, | Sample # | Time Post Infusion, | |
|---|---|---|---|
| Day | EPO906 | hours | EPO906 MI |
| 1 | 1 | Pre-dose* | 2 |
| | 2 | End of infusion for | 2 |
| | | EPO906** | |
| | 3 | 0.5 | 2 |
| | 4 | 1 | 2 |
| | 5 | 2 | 2 |
| | 6 | 4 | 2 |
| | 7 | 10 | 2 |
| 2 | 8 | 24 | 2 |
| 4 | 10 | 72 | 2 |
| 8 | 11 (pre- | 168* | 2 |
| | infusion) | | |
| 22 | 13 (pre- | 504* | 2 |
| | infusion) | | |
| Total | | | 26 |

*This sample must be drawn before EPO906 and oxaliplatin administration.
**This sample must be drawn just prior to or at the end of infusion and the actual time should be recorded.

Samples will be centrifuged immediately at 2500 rpms for 10 minutes at room temperature (25° C.). The plasma layer will be transferred to labeled, polypropylene tubes and frozen at −70° C. The samples will be transported on ice to Novartis where they will remain frozen until liquid chromatographic assays are performed.

### Evaluation for Response

The RECIST criteria for response (see http://ctep.info.nih.gov/) will be followed. All measurable lesions, up to a maximum of 10 lesions, representative of all involved organs, will be identified as target lesions and will be recorded and measured at baseline. Target lesions will be selected on the basis of their size (lesions with the longest diameter) and their suitability for accurate repetitive measurements (either by imaging techniques or clinically). A sum of the longest diameter (LD) for all target lesions will be calculated and reported as the baseline sum LD. The baseline sum LD will be used as reference to further characterize the objective tumor response of the measurable dimension of the disease. All other lesions (or sites of disease) should be identified as non-target lesions and should also be recorded at baseline. Measurements are not required and these lesions should be followed as “present” or “absent”.

Target lesions will be evaluated as follows:

### Complete Response (CR): Disappearance of All Target Lesions

Partial Response (PR): At least a 30% decrease in the sum of LD of target lesions taking as reference the baseline sum LD.
Progression (PD): At least a 20% increase in the sum of LD of target lesions taking as references the smallest sum LD recorded since the treatment started or the appearance of one or more new lesions
Stable Disease (SD): Neither sufficient shrinkage to qualify for PR nor sufficient increase to qualify for PD taking as references the smallest sum LD since the treatment started.

Subjects with measurable or evaluable disease will be assessed at baseline, every two cycles during treatment, and at study termination to obtain any preliminary response data. The indicator tumor can be a primary or metastatic lesion found on diagnostic imaging. The method of measurement used at baseline will be used to follow subjects for response throughout the study. For subjects with bone-only disease, bone scan will be the diagnostic test used to assess response. All subjects will be evaluated with CT scans regardless of location of the indicator tumor.

All responding subjects (CR/PR) will have their response confirmed 4-6 weeks after the first documentation of response. An evaluable drug course will be defined as one in which subjects receive day 1 therapy.

1. A combination of (a) an epothilone and (b) two or more other antineoplastic agents for simultaneous, separate or sequential use.

2. The combination according to claim 1 where the microtubule active agent (a) is Epothilone A or B, or a pharmaceutically acceptable salt thereof.

3. The combination according to claim 1 where the microtubule active agent (a) is Epothilone A or B, or a pharmaceutically acceptable salt thereof, and (b) the two or more antineoplastic agents are selected from topoisomerase I inhibitors, topoisomerase II inhibitors, other microtubule active agents, alkylating agents, antineoplastic antimetabolites, platin compounds, angiostatic steroids, biological response modifiers, monoclonal antibodies, proteasome inhibitors, EGFR inhibitors, leucovorin, interleukin, temozolomide and hexylmethyl-melamine, which are independently of each other in free form or as pharmaceutically acceptable salts.

4. The combination according to claim 1 where the microtubule active agent (a) is Epothilone A or B, or a pharmaceutically acceptable salt thereof, and (b) the two or more antineoplastic agents are selected from Vincristine, Vinblastine, Vinorelbine, capecitabine, Topotecan, Irinotecan, Etoposide, Doxil, doxirubican, Mitoxantrone, Cyclophosphamide, Cisplatinum, Carboplatin, Oxaliplatin, Herceptin, Leucovorin, Radiation, Prednisone, Interluekin, Interferon, Estramustine, 5FU/LV, Dacarbazine, bortezomib, bevacizumab, cetuximab, erlotinib, gefitinib, temozolomide and hexylmethyl-melamine, Gemcitabine, or Procarbazine, which are independently of each other present in free form or as pharmaceutically acceptable salts.

5. Use of a combination which comprises (a) an epothilone and (b) two or more other antineoplastic agents, where the active compounds falling under (a) and/or (b) are independently of each other in free form or in the form of pharmaceutically acceptable salts, for delay of progression or treatment of a proliferative disease.

6. The use according to claim 5 where component (a) is Epothilone A or B, or a pharmaceutically acceptable salt thereof.

7. The use according to claim 5 where component (a) is Epothilone A or B, or a pharmaceutically acceptable salt thereof, and component (b) is a combination of two or more of the compounds selected from topoisomerase I inhibitors, topoisomerase II inhibitors, other microtubule active agents, alkylating agents, antineoplastic antimetabolites, platin compounds, angiostatic steroids, biological response modifiers, monoclonal antibodies, proteasome inhibitors, EGFR inhibitors, leucovorin, interleukin, temozolomide and hexylmethyl-melamine, which are independently of each other present in free form or as pharmaceutically acceptable salts.

8. The use according to claim 5 where component (a) is Epothilone A or B, or a pharmaceutically acceptable salt thereof, and component (b) includes two or more of the compounds selected from Vincristine, Vinblastine, Vinorelbine, capecitabine, Topotecan, Irinotecan, Etoposide, Doxil, doxirubican, Mitoxantrone, Cyclophosphamide, Cisplatinum, Carboplatin, Oxaliplatin, Herceptin, Leucovorin, Radiation, Prednisone, Interluekin, Interferon, Estramustine, 5FU/LV, Dacarbazine, bortezomib, bevacizumab, cetuximab, erlotinib, gefitinib, temozolomide and hexylmethyl-melamine, Gemcitabine, or Procarbazine which are independently of each other present in free form or as pharmaceutically acceptable salts.

9. The use according to claim 5 where the proliferative disease is a solid tumor.

10. A pharmaceutical composition comprising a combination of (a) an epothilone with (b) two or more other antineoplastic agents and optionally at least one pharmaceutically acceptable carrier for the delay of progression or treatment of a proliferative disease.

11. The pharmaceutical composition according to claim 10 where component (a) is Epothilone A or B or a pharmaceutically acceptable salt thereof.

12. The pharmaceutical composition according to claim 10 where component (a) is Epothilone A or B, or a pharmaceutically acceptable salt thereof, and component (b) includes two or three, preferably two, further antineoplastic agents selected from topoisomerase I inhibitors, topoisomerase II inhibitors, other microtubule active agents, alkylating agents, antineoplastic antimetabolites, platin compounds, angiostatic steroids, biological response modifiers, monoclonal antibodies, proteasome inhibitors, EGFR inhibitors, leucovorin, interleukin, temozolomide and hexylmethyl-melamine, which are independently of each other present in free form or as pharmaceutically acceptable salts.

13. The pharmaceutical composition according to claim 10 where component (a) is Epothilone A or B or a pharmaceutically acceptable salt thereof, and component (b) includes two or more of the compounds selected from Vincristine, Vinblastine, Vinorelbine, capecitabine, Topotecan, Irinotecan, Etoposide, Doxil, doxirubican, Mitoxantrone, Cyclophosphamide, Cisplatinum, Carboplatin, Oxaliplatin, Herceptin, Leucovorin, Radiation, Prednisone, Interluekin, Interferon, Estramustine, 5FU/LV, Dacarbazine, bortezomib, bevacizumab, cetuximab, erlotinib, gefitinib, temozolomide and hexylmethyl-melamine, Gemcitabine, or Procarbazine which are independently of each other present in free form or as pharmaceutically acceptable salts.

14. A commercial package comprising (a) an epothilone and (b) two or more other antineoplasfic agents, where the active compounds falling under (a) and/or (b) are independently of each other in free form or in the form of pharmaceutically acceptable salts, for simultaneous, chronically staggered or (less preferably) separate use in the delay of progression or treatment of a proliferative disease.
